(19) United States
(12) Patent Application Publication
(10) Pub. No.: US 2005/0064436 A1
(43) Pub. Date: Mar. 24, 2005

(54) Methods and compositions for identifying patient samples

(76) Inventors: Michael T. Barrett, Mountain View, CA (US); Michael P. Caren, Palo Alto, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/670,862

(22) Filed: Sep. 24, 2003

(51) Int. Cl. C12Q 1/68

(52) U.S. Cl. 435/6

(57) ABSTRACT

Methods and compositions for identifying patient samples are provided. In practicing the subject methods, an SNP profile is determined for a nucleic acid sample, where the determined SNP profile is then employed to identify the source of the sample, e.g., the subject or patient from which the sample was obtained. Also provided are methods of assaying subjects for a condition, e.g., a disease condition, where the assays included an SNP profile based sample source identification step. In addition, compositions and kits for use in practicing the subject methods are provided. The subject methods and compositions find use in a variety of different applications, and are particularly suited for use in clinical assay applications.

### FIELD OF THE INVENTION

The present invention relates to biopolymeric arrays, particularly as employed in clinical assay applications.

### BACKGROUND OF THE INVENTION

Typically the diagnostic process begins with the acquisition of an initial biopsy, such as a blood sample, followed by a series of tests that involve different samples and/or preparations, including materials for archiving. For example a biopsy acquired in an outpatient setting may be used for histology, flow cytometry, somatic mutation screening, and genotyping in baseline and follow-up studies, as well as archival preparation. Furthermore many of these samples will be processed as part of batch sample preparations that involve automated multiple-sample processing for assays that involve different data formats and end points.

In order to assure proper processing of patient samples in the diagnostic process, coded patient identifiers are typically assigned to the patient samples in order to track each sample from its origin while maintaining ethical handling of the samples. A number of methods exist for coding clinical and biological samples. Most of these methods involve attaching a randomly generated unique identifier to patient and biopsy materials in order to assure confidentiality of patient data. Typically these randomly generated unique identifiers involve a random alphanumerical code, such as an Institutional Review Board (IRB) number that is stored in a database at a single clinical site. These codes are typically attached to any sample container or platform (such as blood or cryopreservation vials, pathology slides, paraffin blocks etc.) that contains a sample of interest.

A major challenge for existing systems of identifying and tracking patient samples and related data is the number of patients and biopsies being screened and the number of tests available. The development of DNA-based diagnostics, including comprehensive genotyping and microarray analyses, provides the potential for high throughput diagnostic testing for both germ line and somatic diseases such as cancer, cardiovascular diseases, and diabetes. As DNA-based diagnostics become more prevalent, the current limits of sample tracking and identifying protocols will be exceeded.

Accordingly, there is a need for the development of a new patient sample tracking and identification protocol that is sufficiently robust to be employed with high throughput clinical testing operations, including array-based clinical testing applications. The present invention satisfies this need.

Relevant Literature

Representative references of interest include: WO 02/056030; WO 02/084249; WO 02/33415; WO 02/39120; U.S. Pat. Nos. 6,210,878; and 6,171,793.

### SUMMARY OF THE INVENTION

Methods and compositions for identifying patient samples are provided. In practicing the subject methods, an SNP profile is determined for a nucleic acid sample, where the determined SNP profile is then employed to identify the source of the sample, e.g., the subject or patient from which the sample was obtained. Also provided are methods of assaying subjects for a condition, e.g., a disease condition, where the assays include an SNP profile based sample source identification step. In addition, compositions and kits for use in practicing the subject methods are provided. The subject methods and compositions find use in a variety of different applications, and are particularly suited for use in clinical assay applications.

### Definitions

Unless defined otherwise, all technical and scientific terms used herein have the same meaning as commonly understood by one of ordinary skill in the art to which this invention belongs. Still, certain elements are defined below for the sake of clarity and ease of reference.

A “biopolymer” is a polymer of one or more types of repeating units. Biopolymers are typically found in biological systems and particularly include polysaccharides (such as carbohydrates), peptides (which term is used to include polypeptides and proteins) and nucleic acids, as well as their analogs, e.g., peptide nucleic acids, etc. A “biomonomer” references a single unit, which can be linked with the same or other biomonomers to form a biopolymer (e.g., a single amino acid or nucleotide with two linking groups one or both of which may have removable protecting groups).

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other nucleic acids that are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more components of interest.

The term “array” encompasses the term “microarray” and refers to an ordered array presented for binding to targets in solution, e.g., nucleic acids, peptides and the like.

An “array,” includes any one-dimensional, two-dimensional or substantially two-dimensional (as well as a three-dimensional) arrangement of addressable regions bearing biopolymers, e.g., nucleic acids, peptides, and the like. Where the arrays are arrays of nucleic acids, the nucleic acids may be adsorbed, physisorbed, chemisorbed, photo-induced cross-linked, or covalently attached to the arrays at any point or points along the nucleic acid chain.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more than ten thousand features, or even more than one hundred thousand features, in an area of less than 20 cm² or even less than 10 cm², e.g., less than about 5 cm², including less than about 1 cm², less than about 1 mm², e.g., 100 μ², or even smaller. For example, features may have widths (that is, diameter, for a round spot) in the range from 1 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm.

Array substrates may be flexible (such as a flexible web). When the substrates are flexible, they may be of various lengths including at least 1 m, at least 2 m, or at least 5 m (or even at least 10 m). “Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm. Array substrates may also be reflective and have little or no transparency. The reflectivity may reduce the absorption of the incident illuminating laser light and subsequent heating if the focused laser beam travels too slowly over a region. The substrate may be at least 20% reflective, preferably at least 50% reflective.

Arrays can be fabricated using drop deposition from pulse-jets of either probe precursors, e.g., nucleic acid precursor units (such as monomers), in the case of in situ fabrication, or the previously obtained probes, e.g., a previously produced nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

An array is “addressable” when it has multiple regions of different moieties (e.g., different oligonucleotide sequences) such that a region (i.e., a “feature” or “spot” of the array) at a particular predetermined location (i.e., an “address”) on the array will detect a particular probe sequence. Array features are typically, but need not be, separated by intervening spaces. In the case of an array in the context of certain embodiments of the present application, the “target” will be referenced as a moiety in a mobile phase (typically fluid), to be detected by “probes” which are bound to the substrate at the various regions. However, in certain embodiments, e.g., Comparative Genomic Hybridization (CGH) applications, it may be more appropriate to view the probes as being in solution and the targets being immobilized on the substrate surface.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

By “remote location,” it is meant a location other than the location at which the array is present and hybridization occurs. For example, a remote location could be another location (e.g., office, lab, etc.) in the same building, city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electronic signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, words such as “top,” “upper,” and “lower” are used in a relative sense only.

The term “stringent assay conditions” as used herein refers to conditions that are compatible to produce binding pairs of probes and targets of sufficient complementarity to provide for the desired level of specificity in the assay while being incompatible to the formation of binding pairs between binding members of insufficient complementary to provide for the desired specificity. Specificity means the ability of a probe to selectively bind to its target and may be determined by evaulating specificity ratios between targets and probes, where a specificity ratio is the ratio of net signal from the target-specific oligonucleotide probe to the average of the signals from non-target specific probes, as described in greater detail in U.S. Pat. No. 6,461,816; the disclosure of which is herein incorporated by reference. Specificity ratios of greater than 2 are suggestive of a target-specific oligonucleotide probe of requisite specificity. Specificity ratios greater than 5 are generally interpreted as indicators of a target-specific oligonucleotide probe having good specificity. Conditions that provide for such specificity ratios between known complementary nucleic acids may be viewed as sufficiently stringent to provide for desired specificity. An example of stringent assay conditions is rotating hybridization at 65° C. in a salt based hybridization buffer with a total monovalent cation concentration of 1.5M (e.g., as described in U.S. patent application Ser. No. 09/655,482 filed on Sep. 5, 2000, the disclosure of which is herein incorporated by reference) followed by washes of 0.5×SSC and 0.1×SSC at room temperature. Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based systems are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

By “clinical assay” is meant an assay or test that is performed on a sample obtained from a host or subject in order to provide information on current health or condition, diagnosis, prognosis, treatment, prevention, and/or monitoring of a condition of the host or subject.

### DETAILED DESCRIPTION OF THE INVENTION

Methods and compositions for identifying patient samples are provided. In practicing the subject methods, an SNP profile is determined for a nucleic acid sample, where the determined SNP profile is then employed to identify the source of the sample, e.g., the subject or patient from which the sample was obtained. Also provided are methods of assaying subjects for a condition, e.g., a disease condition, where the assays include an SNP profile based sample source identification step. In addition, compositions and kits for use in practicing the subject methods are provided. The subject methods and compositions find use in a variety of different applications, and are particularly suited for use in clinical assay applications.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context-clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components that are described in the publications that might be used in connection with the presently described invention.

In further describing the invention in greater detail than provided in the Summary and as informed by the Background and Definitions provided above, representative embodiments of the subject methods of sample source identification are reviewed first in greater detail, followed by a discussion of representative applications, e.g., clinical assays, in which the sample identification methods find use, as well as a review of representative compositions, e.g., kits and systems and components thereof, that find use in practicing the subject methods.

Methods

As summarized above, the subject invention provides methods of identifying the source of a sample, particularly a genomic nucleic acid sample. By genomic nucleic acid sample is meant a sample that includes the genomic DNA of an organism. The organism that is the source of the sample may be any of a variety of organisms, where the organisms are typically animals, where animals of interest in many embodiments are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In many embodiments, the hosts or subjects from which the sample is obtained in the subject methods will be humans.

The sample may be any of a variety of different physiological samples that are obtainable from a host or subject, where representative samples of interest include, but are not limited to: whole blood, plasma, serum, semen, saliva, tears, urine, fecal material, sweat, buccal fluid, skin fluid, spinal fluid and hair; in vitro cell cultures, including a growth medium, cells and cell components; tissue biopsies and samples, surgically-excised tissues, and the like. The sample may or may not be pretreated, e.g., by the addition of one or more agents of interest, such as preservatives, chaotropic agents, labeling agents, etc., as is known in the art.

A feature of the subject methods is that a single nucleotide polymorphism (SNP) profile is determined for the genomic nucleic acid sample whose source is to be identified using the subject methods. As is known in the art, a SNP is a change (e.g. a deletion, insertion or substitution) in any single nucleotide base in the region of the genome of interest. Such a base exchange can take place within a gene or in non-expressed areas between the genes, and may or may not be associated with a particular phenotype.

By SNP profile is meant a collection of a plurality of individual SNP data points or values, i.e., a collection of a plurality of identified bases at known SNP locations of a genome of an organism. The number of individual SNP data points or values making up a given SNP profile may vary, and should be sufficiently large to be unique for a specific organism of a given population of individuals, where the population may or may not be the entire species population, and may be only a portion of an entire species population, e.g., from about 10 to 1 million individual or more. In many embodiments, the number of individual SNP values in a given profile is at least about 1, such as at least about 10, including at least about 50, where the number may be a great as 1×10⁷ or greater, but sometimes will not exceed about 10,000, such as about 1,000. The SNP profile may have any convenient data format, such as raw sequence format, processed sequence format, etc.

The SNP profile may be determined for a given sample using any convenient protocol. Representative protocols include both non array-based and array-based SNP detection protocols. As is known in the art, a number of methods are available for analyzing nucleic acids for the presence of a specific sequence, and particularly for the presence of a particular nucleotide base at a known SNP location of a particular sequence. Where large amounts of DNA are available, genomic DNA is used directly. Alternatively, the region of interest is cloned into a suitable vector and grown in sufficient quantity for analysis. The nucleic acid may be amplified by conventional techniques, such as the polymerase chain reaction (PCR), to provide sufficient amounts for analysis. The use of the polymerase chain reaction is described in Saiki et al. (1985) Science 230:1350-1354, and a review of current techniques may be found in Sambrook et al Molecular Cloning: A Laboratory Manual, CSH Press 1989, pp. 14.2-14.33. Amplification may be used to determine whether a polymorphism is present, by using a primer that is specific for the polymorphism. Alternatively, various methods are known in the art that utilize oligonucleotide ligation as a means of detecting polymorphisms, for examples see Riley et al (1990) Nucleic Acids Res 18:2887-2890; and Delahunty et al (1996) Am J Hum Genet 58:1239-1246.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²p, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acids, e.g., amplified or cloned fragment, are analyzed by one of a number of methods known in the art. The nucleic acids may be sequenced by dideoxy or other methods. Hybridization with the variant sequence may also be used to determine its presence, by Southern blots, dot blots, etc. The hybridization pattern of a control and variant sequence to an array of nucleic acid, e.g., oligonucleotide, probes immobilized on a solid support (where representative such arrays are described in U.S. Pat. Nos. 6,599,693; 6,589,739; 6,587,579; 6,420,180; 6,387,636; 6,309,875; 6,232,072; 6,221,653; 6,180,351; the disclosures of which are herein incorporated by reference), may also be used as a means of detecting the presence of variant sequences. In other words, an array-based protocol using an array of SNP specific probe features may be employed to determine the SNP profile of a given sample.

Single strand conformational polymorphism (SSCP) analysis, denaturing gradient gel electrophoresis (DGGE), mismatch cleavage detection, and heteroduplex analysis in gel matrices may be used to detect conformational changes created by DNA sequence variation as alterations in electrophoretic mobility, and these methods may also therefore be used in the determination of a samples SNP profile. Alternatively, where a polymorphism creates or destroys a recognition site for a restriction endonuclease (restriction fragment length polymorphism, RFLP), the sample may be digested with that endonuclease, and the products size fractionated to determine whether the fragment was digested. Fractionation may be performed by gel or capillary electrophoresis, particularly acrylamide or agarose gels. Such an approach provides yet another representative protocol for determining the SNP profile of a sample.

In certain embodiments of particular interest, an array-based protocol is employed to determine the SNP profile of a sample. In these embodiments of the invention, an array of SNP nucleic acid probe features is provided, where discrete positions or features on the array are made up of probe nucleic acids complementary to one or more of the to be screened or detected polymorphic sequences, e.g. oligonucleotides of at least 12 nt, frequently 20 nt, or larger, and including the sequence flanking the polymorphic position. Such arrays may include a series of different nucleic acid features, each of which can specifically hybridize to a different polymorphism. For examples of arrays, see Hacia et al. (1996) Nat Genet 14:441-447 and DeRisi et al. (1996) Nat Genet 14:457-460; as well as U.S. Pat. Nos. 6,599,693; 6,589,739; 6,587,579; 6,420,180; 6,387,636; 6,309,875; 6,232,072; 6,221,653; 6,180,351; the disclosures of which are herein incorporated by reference.

Techniques for SNP detection and analysis are also described in: Sapolsky et al. (1999) U.S. Pat. No. 5,858,659; Shuber (1997) U.S. Pat. No. 5,633,134; Dahlberg (1998) U.S. Pat. No. 5,719,028; Murigneux (1998) WO98/30717; Shuber (1997) WO97/10366; Murphy et al. (1998) WO98/44157; Lander et al. (1998) WO98/20165; Goelet et al. (1995) WO95/12607 and Cronin et al. (1998) WO98/30883. In addition, ligase based methods are described by Barany et al. (1997) WO97/31256 and Chen et al. Genome Res. 1998;8(5):549-56; mass-spectroscopy-based methods by Monforte (1998) WO98/12355, Turano et al. (1998) WO98/14616 and Ross et al. (1997) Anal Chem. 15, 4197-202; PCR-based methods by Hauser, et al. (1998) Plant J. 16,117-25; exonuclease-based methods by Mundy U.S. Pat. No. 4,656,127; dideoxynucleotide-based methods by Cohen et al. WO91/02087; Genetic Bit Analysis or GBA.TM. by Goelet et al. WO92/15712; Oligonucleotide Ligation Assays or OLAs by Landegren et al. (1988) Science 241:1077-1080 and Nickerson et al. (1990) Proc. Natl. Acad. Sci. (U.S.A.) 87:8923-8927; and primer-guided nucleotide incorporation procedures by Prezant et al. (1992) Hum. Mutat. 1:159-164; Ugozzoli et al. (1992) GATA 9:107-112; Nyreen et al. (1993) Anal. Biochem. 208:171-175.

Once the SNP profile for the sample is obtained, e.g., using an SNP determination protocol as described above, the determined SNP profile is then employed to identify the source of the sample for which the SNP profile has been determined. Typically, this identification step is accomplished by comparing the SNP profile to an SNP profile reference, which reference is typically made up of a plurality or collection of distinct SNP profiles, each of which is paired, i.e., linked or matched, to a specific sample source, e.g., an organism (such as a subject or patient) from which the sample was initially obtained. The SNP profile reference may be present in a variety of different formats. For example, the SNP profile reference may be a series of SNP profiles matched with sample sources printed on a printable substrate, e.g., paper. Alternatively, the SNP profile reference may be in the form of a database (which may be searchable) recorded on a suitable computer readable medium, as described in greater detail below.

Comparing the determined SNP profile for a given sample to an SNP profile reference may be accomplished using any convenient protocol. For example, a skilled worked may manually compare the identified SNP profile to an SNP profile reference and thereby identify the source of the sample. Alternatively, the obtained SNP profile may be employed to query a database on a computer readable medium, e.g., by using suitable computer hardware elements, in order to obtain the source of the sample.

Regardless of the particular protocol employed, the above methods result in the identification of the sample source from the determined SNP profile of the sample. The resultant identification may be in the form of one or more actual identification parameters for the source (e.g., patient or subject), such as age, sex, weight, name, etc., or the identification may be in the form of a coded identifier for the subject, so as to maintain the confidentiality of the subject.

Utility

As demonstrated above, the subject invention provides methods of identifying the source of a nucleic acid sample by determining an SNP profile for the sample and then identifying the source of the sample from the determined SNP profile. The subject methods find use in a variety of different applications where source identification of a nucleic acid sample is desired.

As one representative utility, the subject invention finds use as a means of identifying the source of patient samples in clinical testing applications. As such, the subject invention provides methods of using SNP profiles (or SNP signatures) as unique sample (and therefore patient or subject) identifiers in clinical testing applications.

In representative clinical testing applications in which the subject SNP based sample source identification methods find use, the clinical testing applications are generally methods of evaluating a subject or patient for a condition. By condition is meant a physiologic condition or state of a subject. Condition is used broadly to refer not only to disease conditions, but also other physiological conditions that are not necessarily disease conditions, e.g., non-disease physiological conditions, such as metabolic rate, etc.

The term “evaluate” is used herein broadly to refer not only to the diagnosis or detection of a given condition of interest, but also to the monitoring of a condition over a given period of time. As such, in certain embodiments one diagnoses a subject for the presence of a given condition, i.e., to determine whether a subject has a given condition. In yet other embodiments, one monitors or tracks, i.e., watches or observes, the progression of a condition in a subject over a period of time.

The subject or patient evaluated in the subject methods may be a variety of different organisms, but is generally an animal, where animals of interest in many embodiments are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In many embodiments, the hosts or subjects from which the sample is obtained in the subject methods will be humans.

In a given clinical testing application or protocol, the source of the sample being tested may be identified using the subject SNP based identification methods at any time, including multiple times, such as at the same time or at a different time from the assay part of the clinical protocol. As such, in certain embodiments, the sample source will have been identified before the sample is assayed for one or more analytes of clinical relevance. For example, one could identify the source of a sample to confirm that the sample is from the intended source prior to performing the clinical assay on the sample. In this way, one can know that the sample is indeed from the intended source (e.g., patient) prior to running the assay. The sample source identification may be performed just prior to the clinical assay, or may be performed at time significantly prior to the clinical assay, including at the time of initial sample obtainment from the source. For example, a number of different samples may be obtained from a patient and the SNP profile for the particular source determined at the time of sample obtainment and stably associated with the various samples obtained from the patient, e.g., by having the SNP profile printed on a label affixed to a container holding the sample, by having the SNP profile recorded on a computer readable medium stably associated with the sample container; etc., where the SNP profile information may be in human and/or computer readable format.

In yet other embodiments, the sample source is identified at the same time that the sample is assayed for one or more analytes of clinical relevance. For example, at the time of testing for one or more clinical analytes of interest, a given-sample may be divided into two parts, one of which is screened for SNP profile and sample source identification and the other of which is screened for one or more clinical analytes of interest. Alternatively, the sample may be screened simultaneously for an SNP profile and clinical analyte(s) using a device that can screen for both types of analytes at the same time, such as an array of probe features, where the array includes both SNP probe features and clinical analyte probe features.

In yet other embodiments, the sample source is identified at a time after the sample is assayed for one or more analytes of clinical relevance. For example, a clinically assayed sample may be subsequently screened for SNP profile and sample source identification in order to confirm that the sample is from the source it is believed to be from prior to running the clinical assay.

In yet other embodiments, the sample source is identified by the subject methods two or more times during a clinical assay protocol, e.g., before and at the same time as an assay for one or more clinically relevant analytes, e.g., in order to provide a high level of confidence that the sample being assayed is from the source it is believed to be from.

While the subject SNP based sample source identification protocols are suitable for use in any type of clinical assay protocol, they are particularly suited for use with high throughput clinical assay protocols, such as array-based clinical assay protocols. By “array-based” is meant that the assay protocols of the subject invention employ an array (as defined above) to assay or test a given sample. As such, in the subject array-based assays, a sample is contacted with an array and binding complexes on the surface of the array are then detected to provide an assay result, as described in greater detail below. As such, array-based assays are characterized by assaying a sample from the subject of interest with an array, as summarized above. The sample may be any of a variety of different physiological samples that are obtainable from a host or subject, where representative samples of interest include, but are not limited to: whole blood, plasma, serum, semen, saliva, tears, urine, fecal material, sweat, buccal fluid, skin fluid, spinal fluid and hair; in vitro cell cultures, including a growth medium, cells and cell components; tissue biopsies and samples, surgically-excised tissues, and the like The sample may or may not be pretreated, e.g., by the addition of one or more agents of interest, such as preservatives, chaotropic agents, labeling agents, etc., as is known in the art.

As the assays are typically clinical assays, the assays are conducted to provide information on the current health or condition, diagnosis, prognosis, treatment, prevention, and/or monitoring of a condition of the host or subject of interest. In other words, the assays are conducted to detect the presence of and/or determine the stage of, severity of, etc., a condition of the host. The condition may or may not be a disease condition. As such, in certain embodiments, the clinical assay is performed to diagnose the presence of, and/or determine the stage or monitor the progression of, a disease condition. In yet other embodiments, the condition may not be a disease condition, but merely a propensity or predisposition for a disease condition. In yet other embodiments, the condition may not be a classical disease condition, but merely a physiological state that can be detected and/or monitored by array-based assay, e.g., a metabolic rate determination, etc.

The nature of the array-based assays may vary, where the assays may be: genomic assays, in which nucleic acid targets in the sample are hybridized to an array of nucleic acid probes on the array; proteomic assays, in which analytes in the sample are specifically bound to an array of proteinaceous binding agent probes on the array; or other types of array assays using other types of arrays, usually biopolymeric arrays, to detect the presence of one or more analytes of interest in the sample. Arrays of interest include those described above.

In general, assay protocols performed according to the subject methods include the following steps: (1) sample obtainment and (2) assay of the sample. In the first sample obtainment step, a sufficient amount of sample is obtained from the host or subject of interest. In many embodiments, the sample is a fluid sample, where the volume of sample obtained in this step may range from, in fluid volumes, from about a few pL (equaling one or several cells) to about 10 mL (as in a blood sample from a human) to much larger quantities such as blood or urine samples from horse or other large animals, and in the case of tissue samples, from about 1-10 cells(or pgs tissue) to about 10⁶ or 10⁷ cells (ng/μgs tissue) in certain embodiments. The sample is typically obtained and placed in a sample containment means, in which it may then be stored for a period of time, as desired. The period of time during which the sample is stored in this step may vary, where the sample is stored typically for at least about several minutes to about 30 minutes or more, but may frequently be overnight such as at least about a week, where the period of time during which the sample is stored may be as long as a year or longer, such as years, decades or longer, where in certain embodiments the sample may be transported or moved from a first location to a second location.

Following sample obtainment, the array-based assay component of the subject methods is performed, in which the presence of a particular analyte(s) of clinical relevance in a given sample is detected at least qualitatively, if not quantitatively. Protocols for carrying out such assays with arrays are well known to those of skill in the art and need not be described in great detail here. Generally, the sample is contacted with an array under conditions sufficient for the analyte(s) (if present) to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g., through use of a signal production system, e.g., an isotopic or fluorescent label present on the analyte, etc. The presence of the analyte in the sample is then deduced from the detection of binding complexes on the substrate surface.

Specific clinical array-based assay applications of interest include hybridization assays in which a nucleic acid array is employed. In these assays, a clinical sample is first obtained and then prepared, where preparation may include labeling of the target nucleic acids with a label, e.g., a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assay protocols that may be employed in a given clinical array based assay include: simple contact with an array; differential gene expression analysis assays where the sample is compared to a reference; and the like.

Patents and patent applications describing methods of using nucleic acid arrays in various applications, including clinical array diagnostic applications, include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

Patents and patent applications describing methods of using proteomic arrays in various applications, including clinical array diagnostic applications, include: U.S. Pat. Nos. 4,591,570; 5,171,695; 5,436,170; 5,486,452; 5,532,128; and 6,197,599; the disclosures of which are herein incorporated by reference; as well as published PCT application Nos. WO 99/39210; WO 00/04832; WO 00/04389; WO 00/04390; WO 00/54046; WO 00/63701; WO 01/14425; and WO 01/40803; the disclosures of the United States priority documents of which are herein incorporated by reference.

In certain embodiments, the subject methods include a step of transmitting data from the clinical assay step, as described above. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in practicing the methods of the subject invention, the array will typically be exposed to a clinical sample (for example, a clinical sample that has been fluorescently labeled) and the array then read. Reading of the array may be accomplished by illuminating the array and reading the location and intensity of resulting fluorescence at each feature of the array to detect any binding complexes on the surface of the array. For example, a scanner may be used for this purpose, such as the AGILENT MICROARRAY SCANNER device available from Agilent Technologies, Palo Alto, Calif. Other suitable apparatuses and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

As indicated above, the clinical array-based assays to which the sample is subjected in the subject methods may be diagnostic assays, e.g., where the presence of a certain condition, such as a disease condition, is determined; or part of a therapeutic regimen, e.g., to monitor the progression of the disease condition.

The subject methods may be used to detect/monitor any condition whose presence and/or state is associated with a defined biopolymeric, e.g., genomic or proteomic, profile, such that a determined biopolymeric profile can be used to determine the presence or state of the condition of interest. A variety of conditions may be detected and/or monitored according to the subject invention. Representative conditions that are amenable to detection and/or monitoring using array-based assays include, but are not limited to: neoplastic disease conditions, cardiovascular disease conditions, pathogenic disease conditions (such as viral disease conditions), neurological, immune function and the like.

Databases

Also provided are collections or databases of multiple SNP profiles paired with specific source, e.g., patient, identification information. In the subject collections or databases, each constituent SNP profile is paired with its own source, typically patient, identification data. The subject collections or databases typically include at least about 10 different SNP profile/source pairings, where the number of pairings may greatly exceed this number, e.g., 100; 1000; 10,000; 100,000 etc. The subject databases find particular use as references in the subject sample source identification methods.

The databases of the subject invention can be printed or recorded on any convenient substrate. In certain embodiments, the databases are printed onto a printable substrate, e.g., paper, where the information may be in human readable or coded format, including computer readable format. The subject databases can also be recorded on computer readable media, e.g., any medium that can be read and accessed directly or indirectly by a computer. Such media include, but are not limited to: magnetic tape; optical storage such as CD-ROM and DVD; electrical storage media such as RAM and ROM; and hybrids of these categories such as magnetic/optical storage media. One of skill in the art can readily appreciate how any of the presently known computer readable mediums can be used to create a manufacture that includes a recording of the data making up the subject databases.

Systems

Also provided by the subject invention are systems for performing the subject SNP profile based sample source identification methods. The systems include at least one or more elements (e.g., one or more reagents, such as PCR primers, labeled nucleotides, restriction enzymes, arrays with SNP probe features, etc.) for determining an SNP profile for a sample (i.e., SNP profile identification elements, which are any reagents/devices employed to determine an SNP profile) and an SNP profile reference or means for accessing the same. In certain embodiments, the systems may further include one or more elements of a clinical assay or protocol, e.g., an array for use in a clinical assay, and the like. The systems may further include a number of additional components that may find use in a given protocol, e.g., sample preparation reagents, labels, etc., where representative embodiments of such components are described elsewhere.

Kits

Kits for use in methods according to the present invention are also provided. The kits at least include one or more components employed in the SNP identification methods, where representative components are described above. In many embodiments, the kits further include at least a reference or means for accessing the same for use in identifying sample source from an SNP profile determined using the SNP profile determination components provided in the kits.

The kits may further include one or more additional components necessary for carrying out a clinical assay, such as an array, sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the array assay devices for carrying out an array based assay.

In addition, the kits will typically include instructions for using the kit components in a method according to the present invention. The instructions of the above-described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

It is evident from the above discussion that SNP-based genotypes provide unique identifiers that are intrinsic to each patient and all samples obtained therefrom. Genotypes can be read at the time of initial clinical test and included in the patient file. They can be stored at a single secure site and then used to track samples in an anonymous format. The patient identification of a given sample can be verified at any time using readily acquired fresh or archived samples, such as blood. In addition, patient sources can be identified using relatively small amounts of material from a given biopsy allowing repeated verification of the origin of any given sample. The use of SNP genotype-based identifier according to the present invention allows access to the sample and related clinical information to be restricted to authorized clinicians in the context of patient consent. The information need not be included as a label for tissue containers, but may be generated de novo at each test and then verified with any previous results. As such, problems associated with mislabeling of tissue containers and security lapses related to disclosures of patient IRB numbers may be avoided. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of identifying a source of a genomic nucleic acid sample, said method comprising:
(a) determining a single nucleotide polymorphism (SNP) profile for said sample; and
(b) identifying said source of said sample from said determined SNP profile.

2. The method according to claim 1, wherein said identifying comprises comparing said determined SNP profile to an SNP profile reference.

3. The method according to claim 1, wherein said sample is a clinical sample.

4. The method according to claim 3, wherein said method further comprises screening said sample for the presence of at least one analyte of clinical relevance.

5. The method according to claim 4, wherein said SNP profile is determined at the same time as said sample is screened for the presence of at least one analyte.

6. The method according to claim 4, wherein said SNP profile is determined at a time different from the time said sample is screened for the presence of at least one analyte.

7. The method according to claim 6, wherein said SNP profile is determined before said sample is screened for the presence of at least one analyte.

8. The method according to claim 4, wherein said sample is screened for the presence of at least one analyte using an array-based assay.

9. The method according to claim 1, wherein said SNP profile is determined using an array-based protocol.

10. The method according to claim 1, wherein said method further comprises assaying said sample for the presence of at least one analyte if said identified SNP profile matches a predetermined source.

11. A method of screening a genomic nucleic acid sample obtained from a subject for the presence of at least one analyte, said method comprising:
assaying said sample for said at least one analyte; and
determining an SNP profile for said sample to identify said subject.

12. The method according to claim 11, wherein said sample is assayed for said at least one analyte using an array-based assay.

13. The method according to claim 11, wherein said determining step occurs at a time different from said assaying step.

14. The method according to claim 13, wherein said determining step occurs before said assaying step.

15. The method according to claim 11, wherein said determining step occurs at the same time as said assaying step.

16. The method according to claim 11, wherein said determining step is performed using an array-based protocol.

17. The method according to claim 11, wherein said method is a method of evaluating said subject for a condition.

18. The method according to claim 17, wherein said condition is a disease condition.

19. The method according to claim 11, wherein said method is a method of diagnosing a subject for said condition.

20. The method according to claim 11, wherein said method is a method of monitoring a subject for said condition.

21. The method according to claim 11, wherein said subject is a human.

22. A genomic nucleic acid sample having associated therewith a source identifying SNP profile.

23. The sample according to claim 22, wherein said sample is present in a container.

24. The sample according to claim 23, wherein said source identifying SNP profile is present on a label affixed to said container.

25. The sample according to claim 24, wherein said SNP profile is present on said label in a human readable format.

26. The sample according to claim 24, wherein said SNP profile is present on said label is a computer readable format.

27. A kit for use in screening a genomic nucleic acid sample for the presence of at least one analyte, said kit comprising:
(a) an SNP profile identification element; and
(b) an analyte detection element.

28. The kit according to claim 27, wherein said SNP profile identification element comprises a plurality of reagents.

29. The kit according to claim 27, wherein said SNP profile identification element comprises a microarray.

30. The kit according to claim 27, wherein said analyte detection element comprises a microarray.

31. A collection of a plurality of different SNP profiles each paired with a specific nucleic acid source, wherein said collection is recorded on a substrate.

32. The collection according to claim 31, wherein said substrate is a computer readable medium.

33. The collection according to claim 31, wherein said substrate is a printable medium.

34. A nucleic acid array for use in performing an array-based clinical assay of a subject for a condition, said array comprising:
(a) one or more analyte probe features for at least one clinical analyte of interest; and
(b) one or more SNP probe features for use in determining an SNP profile for said sample.

35. A method of performing an array-based condition evaluation assay of a subject, said method comprising:
conducting an array-based assay on a sample believed to be from said subject; and
conducting an SNP profile identification step on said sample to determine whether sample is from said subject.

36. The method according to claim 35, wherein said array-based assay comprises using an array that includes SNP identification probe features.

37. The method according to claim 35, wherein said array-based assay comprises:
(a) contacting said sample from said subject with an array according to claim 34; and
(b) reading said array to detect any binding complexes on the surface of the said array to obtain an assay result.

38. The method according to claim 37, wherein said method further comprises a data transmission step in which a result from said array-based assay is transmitted from a first location to a second location.

39. A method according to claim 38, wherein said second location is a remote location.

40. A method comprising receiving data representing a result of a reading obtained by the method of claim 35.
